(19) United States
(12) Patent Application Publication
(10) Pub. No.: US 2002/0182603 A1
(43) Pub. Date: Dec. 5, 2002

(54) Uniformly functionalized surfaces for microarrays

(76) Inventors: William H. Chapman JR., San Leandro, CA (US); Thuc Le, Milpitas, CA (US); Leonard Klevan, Orinda, CA (US)

Correspondence Address: FISH & RICHARDSON P.C., 500 ARGUELLO STREET, SUITE 500, REDWOOD CITY, CA, 94063, US

(21) Appl. No.: 09/839,588

(22) Filed: Apr. 20, 2001

(51) Int. Cl. C12Q 1/68; G01N 33/53; G01N 33/542; G06K 9/00; A61L 2/00

(52) U.S. Cl. 435/6; 435/7.9; 427/2.11; 382/128

(57) ABSTRACT

Methods for fabricating functionalized substrate surfaces for use in preparing biomolecular microarrays, such that the substrate surfaces feature a uniform distribution of attachment functionality, functionalized substrates having such uniform distribution of attachment functionality, and microarrays prepared from such functionalized substrates. A plurality of linker groups are coupled to a substrate surface. A plurality of spacer groups including attachment sites for biological receptors are coupled to the linker groups. The linker groups can be coupled to the surface using a gas phase reaction. Spacers can include polyfunctional linear, branched or dendritic structures, such as polyethylene glycols and Starburst™ dendrimers. Attachment sites can be activated for the attachment of biological receptors.

### TECHNICAL FIELD

This invention relates to methods and apparatus for preparing microarrays, and more particularly to techniques for preparing substrates for such microarrays having uniformly coated, functionalized surfaces.

### BACKGROUND

The availability of sequence data that describes a majority of the human genome has created a need for higher throughput assays for gene identification and quantification. High throughput, cost effective methods will allow genetic sequence information to eventually be translated into useful descriptions of phenotype, but only after very complete population studies allow for the establishment of empirical genotype-phenotype relationships. Methods that have been used thus far for these types of population studies (e.g., PCR, Southern blots, etc.) allow for the measurement of several (e.g., 1-100) specific gene sequences and variations in those sequences in a single experiment. Since the number of gene sequences present in the human genome is estimated to be 20,000-50,000, it becomes clear that it is necessary to analyze samples from one individual in approximately 200-50,000 different conventional tests in order for its full genetic diversity to be revealed. Since the number of individuals that need to be analyzed is also very large (circa 1,000,000), the number of samples necessary (2×10⁸−1×10¹¹) becomes far too large for a conventional laboratory to process. By increasing the number of sequences or sequence variations detectable in each test, the number of tests necessary to analyze one sample becomes more manageable and large population studies become much more possible.

One method that is currently being developed for these purposes, DNA microarray analysis, allows for the detection and quantification of DNA sequences in μL volumes of sample. Large numbers (circa 10⁵) of DNA hybridization probes are arrayed onto a flat solid surface to which they become permanently attached. The surface is placed in contact with a liquid sample thought to contain some or all of the sequences complementary to those attached to the solid surface and the appearance of hybridization events is used as an indicator of the presence of complimentary sequences in the sample. Methods for the detection of the hybridization events using fluorescence labeling of the samples and fluorescence imaging of the surfaces are known and established. The microarrays are typically fabricated using one of two different methods: 1) by depositing the sample onto a solid (porous or non-porous) surface; or 2) synthesis of a hybridization probe directly on the glass surface (in situ synthesis). The former method is more generally useful because it is possible to deposit nucleic acids of any length onto the surface. The so-called in situ method only allows short fragments to be synthesized and, as a result, has limited utility.

In a typical microarray experiment, each feature (e.g., each nucleic acid fragment or oligonucleotide hybridization probe) placed onto the substrate will have a different sequence. Ideally, this sequence diversity will be the only factor differentiating the thousands of features that cover the substrate surface. In practice, though, variation in the number of points of attachment, the surface charge and other factors may have a strong effect on the behavior of the DNA and may create other differences between the features that, in some cases, may overwhelm the sequence diversity across the surface, undermining the reliability of hybridization results. Thus, it is desirable to minimize any variation in the density of attachment functionality at the surface of materials used as microarray substrates.

Several substrate materials have been reported to be useful in microarray analysis, but have not been designed for specifically for these types of experiments. One such material is poly-1-lysine coated glass, described in Japanese Patent Abstract Publication number 59188541 A and Schena, M., et al (1995) Science 270, 467. Others have reported the preparation of glass functionalized by the reaction of glass surfaces with a silyl group that carries an attachment functionality, as described in Matveev, S. V. (1994) Biosensors and Bioelectronics 9, 333-336. Aminopropylsilane-coated slides have been brought to market by several manufacturers, including Sigma Chemicals of St. Louis, Mo., and Corning Incorporated of Corning, N.Y. Arrays printed onto such slides have been reported to be more reproducible than those printed onto poly-1-lysine. Hegde, P, et al. (2000) Biotechniques 29, 391.

Nevertheless, these microarray fabrication materials and techniques are far from optimized, and in particular have been found to offer substrate surfaces that lack a high degree of surface uniformity in the distribution of attachment functionality, as will be discussed below. Accordingly, there remains a need for microarray substrates that provide a high degree of reproducibility within and between experiments.

### SUMMARY

The invention provides methods for generating substrate surfaces featuring a high degree of uniformity in the surface distribution of attachment functionality, as well as articles produced according to such methods.

In general, in one aspect, the invention features methods for fabricating microarray substrates. The methods include providing a substrate having a surface, exposing the substrate to a concentration of linker molecules in the gas phase under conditions sufficient to couple a plurality of the linker molecules to the substrate surface, and exposing the substrate to a concentration of one or more spacer molecules under conditions sufficient to couple one or more spacer molecules to each of a plurality of the coupled linker molecules to form a functionalized substrate surface. The spacer molecules include one or more attachment sites for coupling a biological receptor to the surface.

Particular implementations of the invention can include one or more of the following features. The functionalized substrate surface can have a uniformity of coverage with attachment sites having a coefficient of variance of less than about 0.25, 0.20 or 0.15. The uniformity of coverage can be determined by exposing the functionalized substrate surface to a concentration of fluorescent reporter molecules under conditions sufficient to couple a plurality of the fluorescent reporter molecules to a plurality of the attachment sites, exciting the fluorescent reporter molecules coupled to the attachment sites and obtaining a fluorescent emission image of the excited fluorescent reporter molecules, and calculating the uniformity of coverage from the fluorescent emission image by calculating the coefficient of variance of the pixel values in the image. The linker molecules can include a functionalized alkyl silane. The linker molecules can include a silane comprising one or more functional groups selected from alkyl halide, amino, thiol, glycidyl, alkene, alkyne, carboxyl, aldehyde, hydrizide, hydroxyl, aryl or heteroaryl groups. The linker molecules can be coupled to the substrate surface through one or more covalent bonds. The spacer molecules can include a Starburst® dendrimer, which can be a poly(amidoamine), poly(amide), poly(urea), poly(carbamate), poly(carbonate) poly(amido alcohol), poly(ether) or poly(thioether). The spacer molecules include a polyethylene glycol. The spacer molecules can include a spacer molecule selected from the group consisting of dendrimers, polyethylene glycols, polyacrylic acid and other vinyl polymers, deoxyribonucleic acids or ribonucleic acids, and amino acid homopolymers. The spacer molecules can have a linear, branched or dendritic structure. The attachment sites can be provided by a functional group such as an amine, amide, ester, ether, thioether, alkyl, alkenyl, alkynyl, aryl or heteroaryl group. The spacer molecules can have a plurality of electrostatic sites for attracting a biological receptor to the surface. The spacer molecules include a histone or a Starburst® polyamidoamine Generation 4 dendrimer. The spacer molecules can be coupled to the linker molecules through one or more covalent bonds. The method can include covalently coupling an activating group to the attachment sites. The activating group can be a photoactivating group, such as an azide containing functional group. The method can include exposing the substrate to a plurality of biological receptors, and activating the activating group to attach a plurality of the biological receptors to the attachment sites.

In general, in another aspect, the invention features functionalized microarray substrates and microarrays prepared by the methods described above. Particular implementations can include one or more of the following features. The functionalized substrate surface can have a uniformity of coverage with attachment sites. The uniformity of coverage can have a coefficient of variance of less than about 0.25, 0.20, or 0.15. The uniformity of coverage can be determined by exposing the functionalized substrate surface to a concentration of fluorescent reporter molecules under conditions sufficient to couple a plurality of the fluorescent reporter molecules to a plurality of the attachment sites, exciting the fluorescent reporter molecules coupled to the attachment sites and obtaining a fluorescent emission image of the excited fluorescent reporter molecules, and calculating the uniformity of coverage from the fluorescent emission image by calculating the coefficient of variance of the pixel values in the image.

In general, in still another aspect, the invention features methods of fabricating a microarray substrate. The methods include providing a substrate having a surface, exposing the substrate to a concentration of linker molecules under conditions sufficient to couple a plurality of the linker molecules to the substrate surface, and exposing the substrate to a concentration of one or more Starburst™ Dendrite spacer molecules under conditions sufficient to couple one or more spacer molecules to each of a plurality of the coupled linker molecules to form a functionalized substrate surface.

In general, in still another aspect, the invention features methods of fabricating microarray substrates. The methods include providing a substrate having a surface, exposing the substrate to a concentration of linker molecules under conditions sufficient to couple a plurality of the linker molecules to the substrate surface, and exposing the substrate to a concentration of one or more polyethylene glycol spacer molecules under conditions sufficient to couple one or more spacer molecules to each of a plurality of the coupled linker molecules to form a functionalized substrate surface.

In general, in another aspect, the invention features microarray substrates. The microarray substrates include a substrate surface, a plurality of linkers coupled to the substrate surface, and a plurality of spacers coupled to the linkers and including one or more attachment sites for coupling a biological receptor to the substrate surface. The microarray substrate has a uniformity of coverage with attachment sites, the uniformity of coverage having a coefficient of variance of less than about 0.25, 0.20, or 0.15. The uniformity of coverage is determined by exposing the functionalized substrate surface to a concentration of fluorescent reporter molecules under conditions sufficient to couple a plurality of the fluorescent reporter molecules to a plurality of the attachment sites, exciting the fluorescent reporter molecules coupled to the attachment sites and obtaining a fluorescent emission image of the excited fluorescent reporter molecules, and calculating the uniformity of coverage from the fluorescent emission image by calculating the coefficient of variance of the pixel values in the image.

Particular implementations of the invention can include one or more of the following features. The linkers can be coupled to the substrate surface through one or more covalent bonds. The spacers can be coupled to the linkers through one or more covalent bonds. The microarray substrates can include a plurality of activating groups coupled to the attachment sites. The linkers can be derived from one or more alkyl silanes, which can include functional groups such as alkyl halide, amino, thiol, glycidyl, alkene, alkyne, carboxyl, oxime, hydrizide, or hydroxyl groups. The spacers can be derived from Starburst® dendrimers, which can be poly(amidoamines), poly(amides), poly(ureas), poly(carbamates), poly(carbonates) poly(amido alcohols), poly(ethers) or poly(thioethers). The spacers can be derived from polyethylene glycol. The spacers can be dendrimers, polyethylene glycols, deoxyribonucleic acids or ribonucleic acids, or amino acid homopolymers. The spacers can include a plurality of electrostatic sites for attracting a biological receptor to the surface.

In general, in another aspect, the invention features microarray substrates and microarrays prepared from such substrates. The microarray substrates include a substrate surface, a plurality of alkylsilane linkers coupled to the substrate surface, and a plurality of Starburst™ Dendrite spacers or polyethylene glycol spacers coupled to the alkylsilane linkers. The microarrays can include a plurality of biological receptors coupled to the spacers.

The techniques of the invention can be advantageously applied to any assay that involves the measurement of a plurality of binding events by the detection of a signal or signals from of a collection of signal generating elements that are chemically bound to a stationary phase. Examples of assays of this type include (but are not limited to): 1) improved quantitation and reproducibility in transcription profiling or related hybridization protocols, 2) simultaneous viral load and mutation screening for viral and retroviral infection therapy; 3) detection and quantification of single nucleotide polymorphisms or other sequence variations in genomic DNA and/or RNA.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows fluorescence scanning images of eleven acylated microarray substrates, including a microarray substrate according to present invention.

FIG. 2 shows fluorescence scanning images of five stained microarray substrates, including a microarray substrate according to present invention.

FIG. 3A is a fluorescence scanning image of a microarray assembled on a microarray substrate according to the present invention after hybridization (532 nm channel on the left, 635 nm channel on the right) to Yeast c-DNA prepared by Reverse Transcription with both Cy3 and Cy5 labeled dNTP.

FIG. 3B is a fluorescence scanning image of a microarray assembled on a commercially available aminopropylsilane substrate after hybridization (532 nm channel on the left, 635 nm channel on the right) to Yeast c-DNA prepared by Reverse Transcription with both Cy3 and Cy5 labeled dNTP.

FIG. 3C is a fluorescence scanning image of a microarray assembled on an aminopropyl silane substrate generated by gas-phase attachment of aminopropyl silane to a glass slide after hybridization (532 nm channel on the left, 635 nm channel on the right) to Yeast c-DNA prepared by Reverse Transcription with both Cy3 and Cy5 labeled dNTP.

FIG. 3D is a fluorescence scanning image of a microarray assembled on a first commercially available poly-1-lysine substrate after hybridization (532 nm channel on the left, 635 nm channel on the right) to Yeast c-DNA prepared by Reverse Transcription with both Cy3 and Cy5 labeled dNTP.

FIG. 3E is a fluorescence scanning image of a microarray assembled on a second commercially available poly-1-lysine substrate after hybridization (532 nm channel on the left, 635 nm channel on the right) to Yeast c-DNA prepared by the Reverse Transcription with both Cy3 and Cy5 labeled dNTP.

FIG. 4 are graphs plotting the signal intensity from Cy3 versus Cy5 from each spot of the arrays illustrated in FIG. 3A, FIG. 3B and FIG. 3D.

Like reference symbols in the various drawings indicate like elements.

### DETAILED DESCRIPTION

The present invention provides functionalized substrates, and methods for preparing such substrates, for use in fabricating microarrays of immobilized receptors suitable for multiplexed biological assays. As used in this specification, receptors include naturally occurring or synthetic molecules having a specific affinity for one or more target ligands. As used in this specification, target ligands can include any molecular species that can be specifically bound by a receptor. Microarrays embodying the techniques described herein can include, for example, arrays of nucleic acids (DNA, RNA), nucleic acid fragments, oligonucleotides, enzymes, antibodies, sugars, polysaccharides, or any other receptor species, and can be useful in assays to determine the presence of specific ligands, including, for example, complementary nucleic acid/oligonucleotide sequences, enzyme substrates, antigens, or the like. Such microarrays, especially those made with the technology described herein, will find applications in a variety of areas including (but not limited to): transcription or translation profiling; simultaneous viral load and mutation screening for viral and retroviral infection therapy; detection and quantification of single nucleotide polymorphisms or other sequence variations in genomic DNA and/or RNA; and highly multiplexed assays for families of proteins (e.g., cytokines) or families of any other biological receptor.

The functionalized substrates of the invention include at least one solid surface of a spatially well-defined chemical nature for the attachment of biological receptors. Suitable substrates can be formed from a variety of materials, which can include, for example, glass, metals (e.g., gold) or metal oxides, and plastics. The choice of a substrate material can in some implementations depend on the particular assay reagents in question as well as the instrumental method chosen for measurement of the signal, and will be apparent to those of ordinary skill in the art. The substrate surface can take a variety of forms, including, for example, flat surfaces, wells, raised regions, etched trenches or the like. As with the choice of particular materials, the choice of a substrate geometry or surface topography can depend on the particular receptor or assay chemistry in question, and will be apparent to those of ordinary skill in the art.

The functionalized substrates disclosed herein provide one or more surfaces featuring a uniform density of functional groups available for coupling to the biological receptors to be arrayed on the surface. As used in this specification, a functional group is a chemical moiety that enables the formation of a covalent bond between the molecule or structure bearing the functional group and another chemical species. Thus, functional groups can include, for example, alcohols, amines, halides, thiols, esters, sulfonates, amides, carboxylates, nitrites, phenols, silanes, activated alkenes, and the like.

The density and distribution of functional groups on a surface can be measured, for example, by a specific chemical reactivity, as will be discussed in more detail below. Biological receptors are bound to the functionalized surface using any of a variety of known techniques. For example, a set of one or more receptors such as nucleic acids, nucleic acid fragments, oligonucleotides, enzymes or antibodies can be reacted with the functionalized surface under conditions conducive to the formation of covalent bonds between the receptor(s) and the surface functionality. Alternatively, the receptors can be assembled directly on the surface functionality—for example, using known solid-phase synthesis techniques.

Preferably, the set of receptors includes a plurality of diverse receptors, such as nucleic acids having different nucleotide sequences, antibodies having different antigen specificity or proteins (such as enzymes and natural or unnatural analogs) having different amino acid sequences. Techniques for introducing such diverse receptors to a functionalized surface are known, including, for example, forming covalent bonds between the receptor and spacer using amide or thioether bonds. This attachment chemistry can be similar to that commonly used for the formation of bonds between receptors and label molecules (fluorescent dyes, enzymes), such as the reactions of activated esters of carboxylic acids with amines, or the addition of thiols to maleimides. The selection of a particular type of attachment chemistry may depend on the particular spacers, receptors and assays involved, as would be recognized by those of ordinary skill in the art. Thus, the particular chemistry by which the receptors are attached to the functionalized substrate surface is not critical to the invention. For example, the UV irradiation of a solid DNA sample deposited onto poly-1-lysine coated glass slide is sufficient for fabrication of a useful array as is described by Hegde, P. et al. (2000) Biotechniques 29, No. 3, 548. Ideally, printing an equal volume of a liquid sample of different biological receptors onto the functionalized surface in different spatial positions produces essentially equal numbers of bound receptors at all positions. This results in microarrays having high reproducibility in binding of biological molecules at a multiple printed site across the array.

Chemical functionality is introduced onto a substrate surface using known chemical methods such as silylation of glass. In one embodiment, the desired uniform distribution of functional groups is obtained by performing an initial functionalization step in which the substrate surface is exposed to a linker molecule in the gas phase. As used in this specification, a linker is a chemical species having at least one functional group capable of forming a physical connection (e.g., a covalent bond) with the substrate surface, and at least one additional functional group suitable for further chemistry as will be discussed below. Suitable linker molecules include, for example, alkyl silanes bearing one or more reactive functional group such as alkyl halide, amino, thiol, glycidyl, alkene, alkyne, aldehyde, oxime, hydrizide, hydroxyl, hetrocyclic and aromatic systems.

In a preferred embodiment, the surface functionality introduced by the attachment of a linker molecule to the substrate surface is extended by attaching an additional spacer to the linker functionality. In particular embodiments, spacers can serve a variety of functions. Preferably, spacers take the form of a bifunctional, multifunctional or polyfunctional molecular species capable of both binding with one or more functional groups on the attached linker molecules and of presenting one or more functional groups providing a framework for the attachment of the desired receptor or receptors to the substrate surface. In particular embodiments, the attachment framework can be provided by one or more functional groups including, but not limited to amines, amides, esters, ethers, thioethers, alkyls, alkenyls, alkynyls and aryl groups.

Thus, a spacer acts to physically connect the linker (and the substrate) to one or more receptors for use in a microarray assay. In general, the presence of a spacer allows the receptor to connected some distance from the surface, such that the receptor's behavior may more closely approximate its behavior in fluid solution, which is generally better understood then receptor behavior at an interface. This permits assay designers to more easily apply known principles to the design of microarray assays. In addition, in some embodiments spacers may provide a three dimensional environment at the receptor-substrate interface that presents more receptor binding functionality per unit area then a flat surface would. Similarly, a three-dimensional spacer environment may favor the binding of the target ligand to the receptor, and may favor the existence of such bound species. For these purposes, spacers can be provided in a variety of architectural forms, including, but not limited to, linear, branched and dendritic forms. Those skilled in the art will recognize that different spacers will be appropriate depending on the particular circumstances of a given use.

In some embodiments, spacers may also provide electrostatic sites that can facilitate the binding of receptor to substrate and/or target ligand to receptor. Examples of such spacers may include histones or other charged proteins, or more generally, polyfunctional polymers bearing multiple charged sites. In these embodiments, coulombic forces derived from charged sites on the spacer may attract charged receptors to the substrate surface, so that the receptor is in close proximity to the spacer. Similarly, such forces can attract charged ligand species, so that the substance to be analyzed is in close proximity to the receptor. In a preferred embodiment, when the receptor is a DNA, for example, charged spacer sites may help to modulate the stability of a DNA duplex structure formed between a single stranded DNA receptor and a complementary DNA target ligand in solution. This modulation can help to control the specificity of this hybridization event, similar, in principle, to controlling the melting behavior of duplex DNA in solution by adding appropriate concentrations of monovalent ions.

Suitable spacer molecules can include, for example, dendrimers, polyethylene glycols, deoxyribonucleic acids or ribonucleic acids, and amino acid homopolymers, such as poly-1-aspartate, poly-1-lysine, or the like. In a preferred embodiment, the spacer is a polyfunctional dendrimer spacer. As used in this specification, a dendrimer is a branching polymer or oligomer that is built generationally from a central core. A preferred dendrimer spacer is a “Starburst®” dendrimer, which is a three-dimensional, highly ordered oligomeric or polymeric compound formed by reiterative reaction sequences starting from a central core. See, e.g., U.S. Pat. No. 4,568,737. Starburst® dendrimers are characterized by discrete, controllable molecular architectures, which makes them particularly well-suited to serve as spacers in the present invention because they can be expected to vary little, if at all, from batch to batch, thus minimizing reproducibility problems in the production of microarray substrates due to variations in spacer architecture. Dendrimers can also incorporate a variety of functional groups suitable for use in the spacers of the present invention, including, for example, poly(amidoamines), poly(amides), poly(ureas), poly(carbamates), poly(carbonates) poly(amido alcohols), poly(ethers) and poly(thioethers).

A particularly preferred dendrimer spacer for use in the present invention is Starburst® polyamidoamine (PAMAM) Generation 4, which is commercially available from Aldrich Chemical Company. This spacer carries both primary amino groups, which can be used to attach the spacer to both the substrate surface and the receptor molecule, and tertiary amino groups that, in the protonated state, can provide an attractive coulombic force that can help to attract a negatively charged receptor (or target ligand), such as a DNA strand, to the surface.

As described above, spacers provide an attachment framework for the attachment of receptors to the substrate surface. In some embodiments, the attachment of receptors to the spacers can be facilitated by activating the attachment functionality with activating groups using known techniques. Appropriate activating groups can include, for example, reactive azide groups, such as aryl azides, phosphorylazides, alkylazides, sulfonylazides. One preferred set of activating groups includes the photosensitive nitroaryl azides such as 5-azido-2-nitrobenzamide groups. As is well-known by those of ordinary skill in the art, the nitroarylazide group is photoactived upon exposure to blue light (λ>310 nm) to produce a reactive nitrene capable of forming, a covalent bond with nearby species, such as a receptor that has been introduced to the surface, by means of a rapid insertion reaction. Because nucleic acids do not absorb light of wavelength greater than 310 nm, the use of such activating groups provides a means for the efficient attachment of nucleic acid receptors to the functionalized surface without interfering with the receptor's ability to hybridize to a complementary nucleic acid strand. Alternative activating groups can include, for example, those derived from Traut's reagent (2-iminothiolane), which react with primary amino groups to yield a charged amidine linkage that carries a free thiol group. These free thiol groups can be used to attach receptors to the surface using, for example, disulfide bonds. Those skilled in the art will recognize that other activating groups may be appropriate for other receptor-ligand systems, and that the selection of a particular activating group will depend on the circumstances of a particular implementation, including such factors as the type of attachment functionality and the nature of the receptor to be attached.

In a preferred embodiment, a functionalized microarray substrate as described above can be prepared by modifying the substrate surface (e.g., the surface of a glass slide) by silylation with, e.g., a chloropropylsilane linker, and displacing the halide with primary amino group carried by a polyamine spacer, as shown in Scheme 1, below. Glass microscope slides (e.g., from Corning Inc. of Corning, N.Y.) are washed with sodium hydroxide in a mixture of ethanol and water, and, after washing with water, the slides are dried by centrifugation and heating. After drying, the slides are mounted in a slide rack, which is placed in a glass desiccator over a quantity of liquid 3-chloropropyltriethoxysilane. The desiccator is evacuated and sealed. After 24 hours at room temperature, the desicator is opened and the slides racks are washed in acetone several times. The chloride is displaced with iodide in acetone and the slides are washed free of the excess NaI with acetone. The iodide is displaced with the polyamine (Generation 4 Starburst® Dendrite) using methanol as a solvent and the slides are washed in methanol to remove any unbound dendrite. Optionally, the resulting polyamine surface is activated by reacting the functionalized surface with a photolabile activating agent such as the N-hydroxysuccinimide ester of 5-azido-2-nitrobenzoic acid in DMF to yield an activated surface acylated with the nitroaryl azide. The slides are dried in an air oven and stored in a sealed container at room temperature. Slides bearing photosensitive activating groups such as the nitroarylazide group are handled only in dim room light.

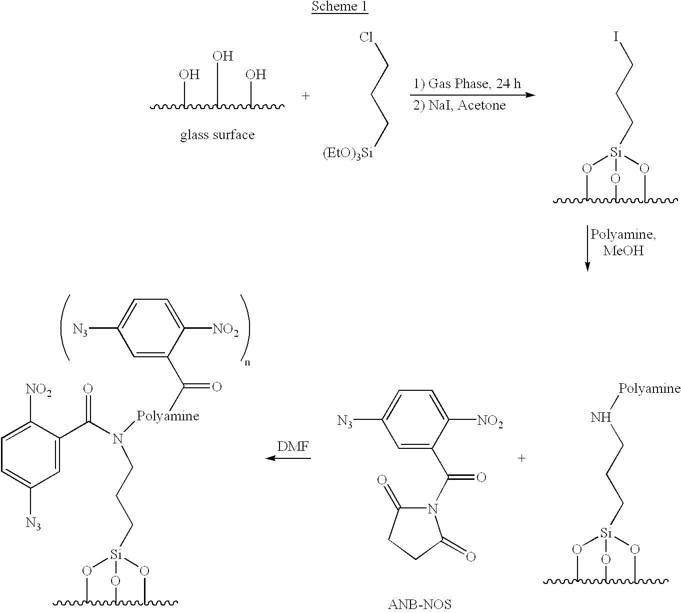

The generation of microarray substrates incorporating the linker and spacer chemistry described herein results in substrates having a high degree of uniformity in the distribution of surface functionality for the attachment of receptor molecules. The density and uniformity of surface functionality can be measured using, for example, fluorescence techniques, by attaching a fluorophore to the surface-bound functional groups, and then imaging the modified surface using a rastering-type fluorescence imaging system. In one embodiment, an aminated glass surface is acylated with a fluorophore such as a Rhodamine NHS ester according to Scheme 2, below. After complete washing, the surface is imaged by fluorescence scanning using a sensitive fluorescence imaging system such as the Hitachi Genetic Systems FMBIO II available from MiraiBio Inc., of Alameda, Calif. Pixel values for the individual slides are extracted from the resulting images and converted into spreadsheet format (using known image processing techniques), and the mean average and standard deviation of the pixel values is determined and used as a measure of surface uniformity (for example, in the form of a coefficient of variance cv=standard deviation/mean average). Functionalized substrate surfaces generated according to the techniques described herein will typically feature coefficients of variance determined in this fashion of less than about 0.30, preferably less than about 0.25, more preferably less than about 0.20 and even more preferably less than about 0.15.

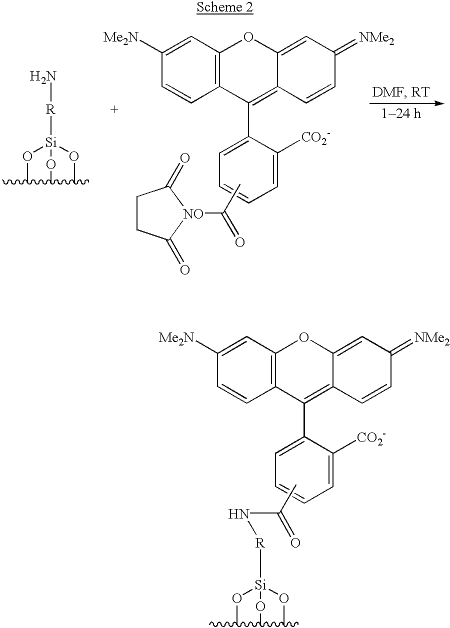

The methods and apparatus described herein are further illustrated and described in the following detailed examples. These examples are offered to further illustrate the various specific and illustrative embodiments and techniques described, and should not be construed to limit the invention to the particular aspects described herein.

### EXAMPLES

Reagents were obtained from the Sigma-Aldrich Chemical Company and were used as received unless otherwise noted. Fluorescence imaging was performed using a Hitachi Genetic Systems FMBIO II fluorescence imaging system. All water used was purified by a two stage process: 1) passage through a Millipore RX-20 unit and 2) passage through a Millipore MilliQ unit. Microscope slides racks and glass staining jars (assembly #121) were purchased from Shandon Lipshaw (Pittsburg, Pa.). All manipulations of the microscope slides were preformed while that slides were mounted in the rack from Shandon. The slides were dried by centrifugation (Beckman Allegra 6R) by placing a rack of slides in a Beckman microplus micro plate carrier on top of a paper towel and centrifuging the rack (with a suitable balance) at 500 RPM and 15° C. for 5 minutes. Slides were washed in either water or organic solvents by lifting the rack up and down quickly by hand (˜1 minute) using the metal clip that was provided with the rack by Shandon Lipshaw.

Glass microscope slides (Corning Incorporated, Corning, N.Y.) were washed for two hours with a mixture of NaOH, ethanol and water with orbital shaking at circa 60 RPM. The NaOH (70 g) was dissolved in water (280 mL) and, after cooling to room temperature, diluted with ethanol (420 mL). The slides were washed four times in water (400 mL/wash) and the slides were dried by centrifugation and finally by heating to 45° C. in an air oven for 15 minutes.

### EXAMPLE 1

### Preparation of the Starburst® Dendrite (PAMAM) Coated Glass

After drying as described above, slides were coated with chloropropylsilane by reaction with gaseous 3-chloropropyltriethoxysilane. The slides were placed in a glass desiccator (Pyrex Brand 3120-250) over 50 mL of 3-chloropropyltriethoxysilane and the desiccator was evacuated and sealed. After 24 hours at room temperature, the desiccator was opened and the slides were washed in acetone three times. The propyl chloride was converted to a propyl iodide by reaction with sodium iodide in acetone. NaI (70 g) was dissolved in acetone (700 mL) and the slides were agitated with the solution for 24 hours with the orbital shaker (60 RPM). The slides were washed twice with acetone (400 mL/wash) and allowed to dry at room temperature. The iodide was displaced with the Starburst® dendrite in methanol. The Starburst Dendrite (Generation 4, 10% weight solution in methanol) was diluted in methanol to give a 0.1% weight % solution. The slides were agitated with the Dendrite solution for 48 hours with orbital shaking. The slides were washed in methanol (400 mL/wash) three times to remove any unbound Starburst® dendrite. The slides were dried in an air oven at 45° C. for 15 minutes and stored in a sealed container at room temperature.

### EXAMPLE 2

### Preparation of Aminopropylsilane Slides By Silation in the Gas Phase

Glass slides, washed and dried as is described above, were placed into a vacuum oven that was prewarmed to 45° C. together with a beaker of 3-aminopropyltrimethoxysilane. The oven was sealed, evacuated and the slides were allowed to react with the silane for 24 hours. After this time, the slides were washed with acetone twice, air dried and then heated to 70° C. for 2 hours. The slides were stored in the dark at room temperature.

### EXAMPLE 3

### Detemination of Surface Uniformity of Acylated Slides

Surface uniformity for five amine-functionalized slides was measured according to Scheme 2, above. Slide one was a Starburst® Dendrite (PAMAM) coated glass slide prepared according to Example 1. Slide two was a commercially available CMT-GAPS aminopropyl silane slide from Coming Incorporated. Slide three was a glass slide that had been coated with aminopropyl silane according to Example 2. Slides four and five were commercially-available poly-1-lysine coated slides from Cel Associates (Houston, Tex.) and Giaman (DNA Chip Reseach, Yokohama, Japan) respectively.

Surface amino groups for each slide were acylated with the NHS ester of Rhodamine according to Scheme 2 as follows.

The NHS-Rhodamine (5-and 6-carboxytetramethylrhodamine, succinimidyl ester, 25 mg, Pierce Chemical Co., 46102) was dissolved in DMF (75 mL, Aldrich, anhydrous) and divided evenly into three plastic microscope slide holders. Microscope slides (three of each type) were labeled by etching with the diamond tipped pin and were submerged in the Rhodamine solution. After five slides had been placed into one container, the plastic cap was closed and sealed with para-film. The solution was agitated by inversion of the container five or six times and the solutions were protected from the room light with an inverted brown cardboard box. After approximately 1 hour, the containers were agitated again and allowed to sit overnight (17 h) at room temperature. The next day, the Rhodamine solution was decanted from the slides, fresh DMF was added and the solution was agitated by inversion 10-15 times. The solution was decanted and the process was repeated once again. The contents of all three of the slide holders were transferred to a metal rack (15 slide capacity) and the rack was washed in DMF (500 mL) twice (20 minutes per wash, occasional agitation). The rack was blotted dry with a towel and washed 3×with 50 mM tris, pH=8 for 10 minutes, spun dry in the centrifuge and placed in a black microscope box for storage before imaging.

The acylated slides were imaged with the Hitachi FMBIO II fluorescence scanner. The slides were mounted in the scanning area such that the laser traces lines in a direction perpendicular to the long dimension of the slide. The slides were scanned for Rhodamine (585 nm filter, 0.8 mm focusing point). Images were analyzed by: 1) extracting the image pixel values corresponding to the individual slides from the fluorescence image; 2) converting the fluorescence pixel values to an Excel (Microsoft Corporation) spread sheet format; and 3) calculating the mean average and standard deviation for all numbers in the Excel spread sheet (using standard Excel functions). Both the extraction and digital conversion steps were accomplished using a software utility for displaying a desired portion of a 16-bit tiff image file in the form of an Excel spread sheet. The final results are presented as coefficients of variance as is defined by the following equation: % c.v.=(standard deviation/mean average)×100.

Results for the five slides are shown in Table 1. An image of an acylated slide with a perfectly even distribution of amino group density on the surface would show a c.v. equal to zero and would be considered to be perfect in this respect for printing arrays. Higher values of the calculated c.v. are indicative of surfaces that have irregularities and would be less than perfect for array production.

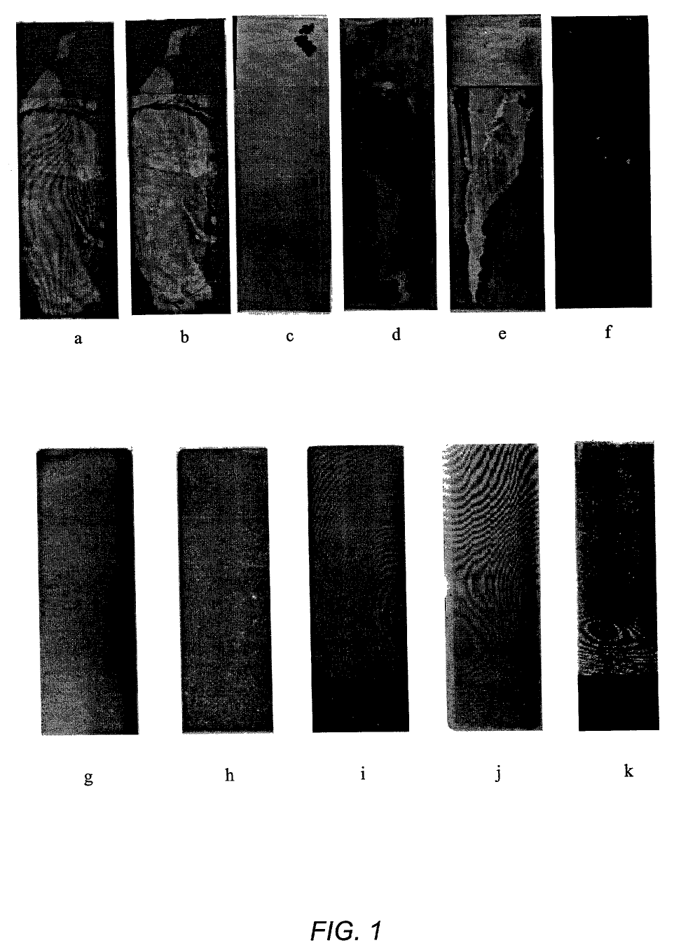

The corresponding images of the surfaces of slides one through five are shown in FIG. 1. Slides are labeled as follows: a, b) poly-1-lysine (prepared according to the protocol of Brown et al., as described http://cmgm.stanford.edu/pbrown/protocols/1_slides.html, by dipping glass slides in basic EtOH/water mixture (50 g NaOH, 150 mL H2O, 200 mL EtOH) for more than 2 hours, washing slides in water 3-4 times, dip slides into poly-1-lysine solution (50% solution from Sigma #8920, diluted to 10% with water) for more than 2 hours, and placing glass slides in microscope slide holder (such that the surface to be printed onto is parallel to the direction of the centrifugal force) and centrifuge at 700 rpm for 1 minute to dry); c, g) APS (Corning, from two different batches); d) APS (silylated in toluene according to the protocol of Matveev, et al., Biosensors & Bioelectronics 9 (1994) 333-336, by washing glass slides as described above for the preparation of the poly-1-lysine slides and drying by centrifugation (dried slides were stored at room temperature); 3-aminopropylsilane (20 mL) was diluted in toluene (180 mL), mixed completely and transferred to a microscope slide staining jar (Aldrich, Z10,397-7); slides were added and allowed to sit at room temperature for 3 hours; slides were removed from the silane containing solvent and immediately washed in the solvent that was used for the silylation in an identical jar; washing was repeated 3 times and the slides were incubated, in the same container, with water for three hours; slides were dried by centrifugation and stored at room temperature); e) silylated with 3-(2-aminoethylamino)-propyltrimethoxysilane in acetone (according to the protocol described for slide d); f, h) PAMAM Starburst™ Dendrite prepared according to Example 1;i) APS prepared according to Example 2;j) poly-1-lysine (Cel Associates, Houston, Texas); k) poly-1-lysine (Giaman, Yokohama, Japan). Images a-f and g-k were produced in different experimental runs. The images of each slide are gray scale corrected to emphasize the evenness of the fluorescence.

TABLE 1

| Slide No. | Slide Type | % c.v. acylation |
|---|---|---|
| 1. | Dendrite (PAMAM) | 12.2 |
| 2. | Corning CMT-GAPS | 28.3 |
| 3. | APS | 11.0 |
| 4. | PLL Cel Associates | 24.4 |
| 5. | PLL (Giaman) | 11.9 |

From the average fluorescence intensity, the average density of the functionality on the surface can be calculated from the appropriate calibration plot as described in Example 9, below. The average density of slide h (PAMAM) shown in FIG. 1 was calculated to be 4497 amino groups per μm².

### EXAMPLE 4

### Preparation of Slides Printed with PCR Products

DNA microarrays were prepared using five different functionalized slides. Again, slide one was a Starburst® Dendrite (PAMAM) coated glass slide prepared according to Example 1. Slide two was a commercially available CMT-GAPS aminopropyl silane slide from Coming Incorporated. Slide three was a glass slide that had been coated with aminopropyl silane according to Example 2. Slides four and five were commercially-available poly-1-lysine coated slides from Cel Associates (Houston, Tex.) and Giarnan (Yokohama, Japan) respectively.

Each of the slides was printed with a set of PCR products derived from the Yeast test pattern, a collection of yeast genes assembled by Hitachi Software's DNA Chip research laboratory that have been found to amplify by PCR to yield well-defined bands on an agarose gel. Samples were provided by Takeshi Sasayama (Hitachi Software Engineering, Yokohama Japan) in the form of PCR plates (96 well) that contained DNA that had been precipitated and dried. The precipitated DNA was dissolved in water to 0.25 μg/μL (20 μL water in 100 μL PCR reaction). The samples (as little as 3 μL) were transferred to 384 well plates as is described below and were mixed with an equal volume of spotting buffer (2×). Samples were printed onto the coated glass slides with four SPBIO pins (4.5 mm spacing, 100 micron pins) from a the 384 well plate. The DNA was arrayed into the plate such that each of the four blocks printed would be identical. Although many different buffers have been found to be compatible with the SPBIO pins, 20% glycerol in TE is preferred. After printing was complete, glass slides were heated at 80° C. for 1 hour in an air oven. Slides were hydrated by incubation at approximately 50° C. in a sealed container with a moist paper towel for 1-5 minutes. The slides were then UV irradiated (120 mJ/cm²). The slides were then blocked by reaction for 12 minutes with succinic anhydride solution prepared by dissolving succinic anhydride (2.5 g) in n-methyl-2-pyrrolidinone (157.5 mL), and adding 0.2 M sodium borate buffer (17.5 mL; pH=8, prepared by the titration of boric acid (0.2 M) with NaOH) immediately after the solid anhydride has dissolved. The slides were immediately washed with water two times, and added to boiling water and denatured for 2 minutes in a microwave oven with a rotating stage. The slides were then washed in ethanol (100%) and are dried at room temperature.

### EXAMPLE 5

### Staining of the DNA Arrays with POPO-3

DNA arrays prepared according to Example 4 were stained for analysis as follows. POPO™-3 iodide stain (1 mM in 200 μL DMF, Molecular Probes, Inc., Eugene, Oreg., www.probes.com) was diluted 10,000-fold in TBE buffer (45 mM Tris-borate, 1 mM EDTA, pH 8.0). The microarrays were submerged in the diluted stain and incubated at room temperature for 2-3 minutes. The microarrays were then washed 3-4 times with the buffer, and were spun dry for 1-2 minutes in a centrifuge.

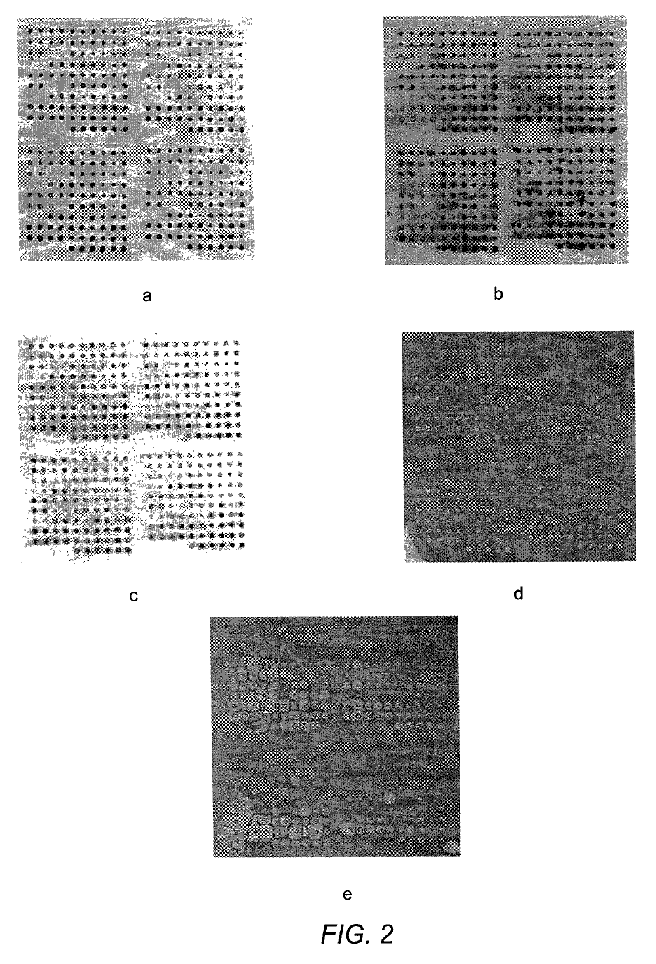

The microarrays were imaged using a GenePix 4000b microarray scanner (Axon Instruments, Union City, Calif.). The resulting spot intensity data was into an Excel spreadsheet and coefficient of variance values calculated for each slide as described in Example 3, above. Results for the five slides are shown in Table 2. Corresponding images of the surfaces of slides one through five are shown in FIG. 2. As FIG. 2 illustrates, slides 3 through 5 performed too poorly to be analyzed.

TABLE 2

| Slide No. | Slide Type | % c.v. POPO3 |
|---|---|---|
| 1. | Dendrite (PAMAM) | 15.7 |
| 2. | Corning CMT-GAPS | 23.4 |
| 3. | APS | N/C |
| 4. | PLL Cel Associates | N/C |
| 5. | PLL (Giaman) | N/C |

### EXAMPLE 6

### Hybridization of Arrays with Dye-Labeled cDNA

DNA arrays prepared according to Example 4 were hybridized to mixtures of dye labeled cDNA (Cy3 and Cy5) prepared from Yeast m-RNA.

cDNA's were prepared as follows. T₁₈ is (1 μL, 0.5 μg/μL) from Operon Technologies (Alameda, Calif.) and the m-RNA sample (2 μg per reaction isolated from Saccharomyces cerevisiae strain DBY746, Clontech catalogue number 6999-1) were added to a total of 7 μL H₂O containing 25 μg e-coli RNA. The samples were incubated at 70° C. for 5 minutes and 42° C. for 2 minutes. To this solution was added: 5× SuperScript II reaction buffer (4 μL); dNTP mix (2 mM TTP, 5 mM dATP, dGTP, dCTP, 2 μL); Cy labeled UTP (1 mM, 2 μL); DTT (100 mM, 2 μL); and RNaseOUT Ribonuclease Inhibitor (LTI, 10777-019, 100 units, 2.5 μL) to yield a total solution volume of 19.5 μL. 1 μL (LTI, 18064-022, 200 U) of SuperScript II Reverse Transcriptase was added, and the mixture was incubated for 30-40 minutes at 42° C. 0.5 μL (100 U) of SuperScript II Reverse Transcriptase was then added, and the mixture incubated for 30-40 minutes at 42° C. To this mixture was added, in this order, 20 μL of H₂O, 5 μL of 0.5 M EDTA and 10 μL 1 M NaOH, and the mixture was incubated at 65° C. for 60 minutes. 25 μL 1 M TrisHCl (pH=7.5) was added to neutralize the reaction.

The dye labeled c-DNA (Cy3, Cy5) samples were purified by membrane filtration (Microcon-30). The solution was concentrated to 10-20 μL, and 250 μL of TE buffer was added. The solution was again concentrated to 10-20 μL. This procedure was repeated 2-3 times to insure complete removal of the remaining triphosphates. After purification, the efficiency of the labeling of the c-DNA with Cy3 or Cy5 can be calculated from the data contained in an absorbance spectrum. The concentration of the dye is calculated from the absorbance of the dye at its maximum (Cy3 at 550 nm and Cy5 at 649 nm) and its extinction coefficient at the same wavelength (Cy3 (550 nm)=150000 M-1 cm-1 and Cy5 (649 nm)=250000 M-1 cm-1). The concentration of nucleic acid bases is calculated from the absorbance of the sample at 260 nm and the average extinction coefficient of a nucleic acid base (circa 10950 M⁻¹cm⁻¹). The Cy dyes do not absorb appreciably at 260 nm. The absorbance of small volumes cDNA samples can be measured using the GeneSpec III absorbance spectrometer, available from MiraiBio, Inc. (www.MiraiBio.com).

The dye-labeled cDNA were hybridized to the microarray slides as follows. 6.25 μL 20×SSC and 1.25 μL 10% SDS were added to a dye labeled cDNA sample, and the mixture was diluted to a final volume of 25 μL. If a precipitate formed during the addition of the SDS, the solution was diluted to the final volume and heated to 37° C. until it cleared. The solution was heated to 95° C. for 2-3 minutes and immediately cooled in ice water. The entire hybridization solution was transferred onto the array with a hand pipette, making sure not to touch the array with the pipet tip. The solution was covered with a glass cover slip, positioned such that the entire DNA array is in contact with the hybridization solution, being careful not to create bubbles between the cover slip and the glass slide. The array was placed in an air-tight container (Tupperware) containing a wet paper towel (insufficient humidity in the container will cause the hybridization buffer to evaporate, leading to the generation of excess background signals), which was incubated at 60-65° C. for >10 hours. After the hybridization was complete, the slides were dipped into 2×SSC, 0.1% SDS solution and the cover slips removed. Each array was washed twice in 2×SSC, 0.1% SDS solution for 20 minutes each at room temperature, then twice in 0.2×SSC, 0.1% SDS for 20 minutes at room temperature, then twice at 40-60° C. in 0.2×SSC, 0.1% SDS for 20 minutes, and finally twice in 0.2% SSC, 0.1% SDS for 20 minutes at room temperature. The arrays were rinsed briefly with 0.05×SSC at room temperature, centrifuged at 600 rpm for 20 seconds, and dried at room temperature for a few minutes.

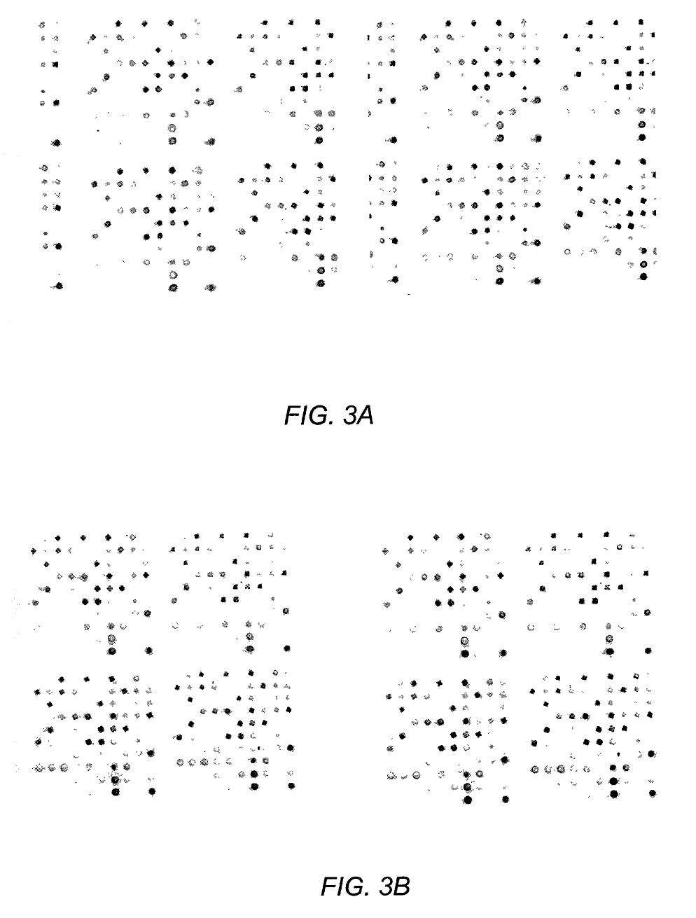

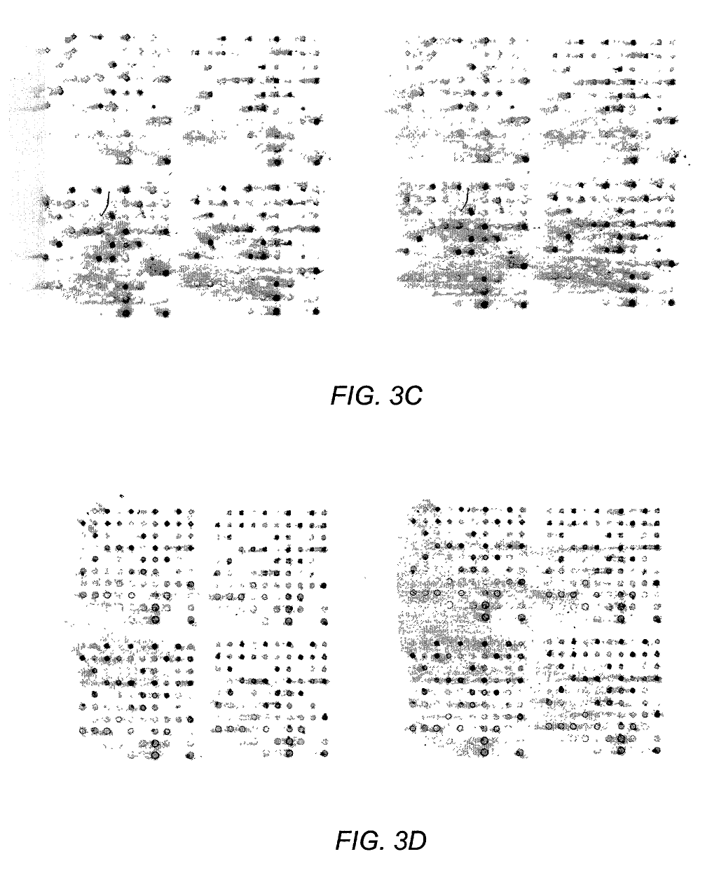

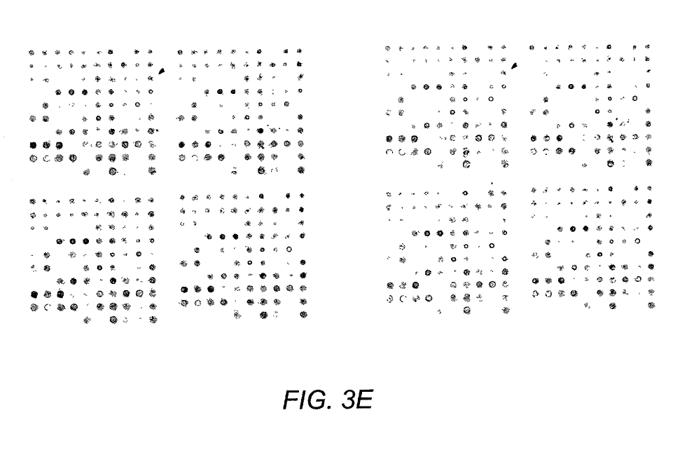

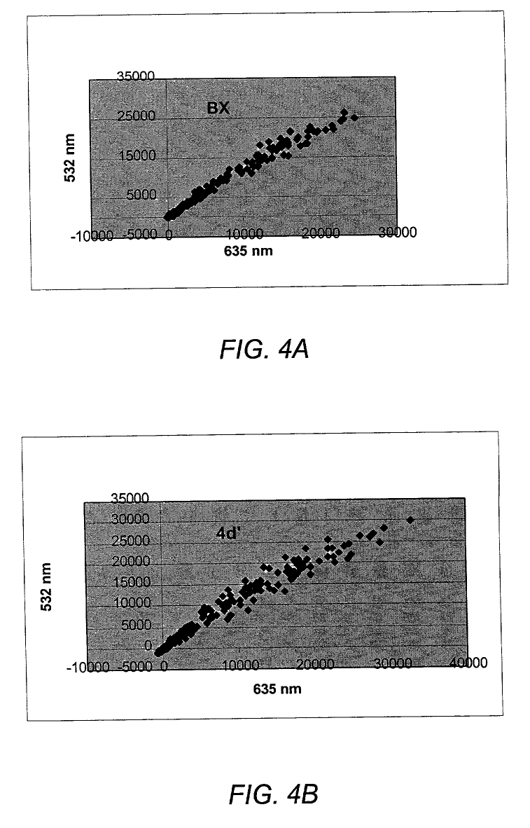

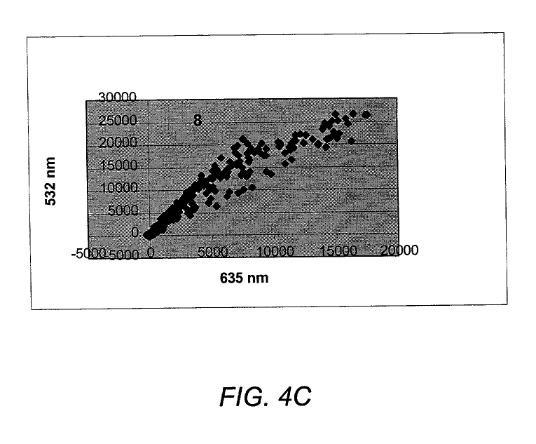

The arrays were then imaged with a GenePix 4000b microarray scanner (Axon Instruments), as described above. Results for the five slides are shown in Table 3. Corresponding images of the surfaces of slides one through five are shown in FIGS. 3A-3E. The error in the array experiments was calculated from the images by: 1) determining the correlation coefficient for a plot of the Cy3 and Cy5 fluorescence intensities of each spot; and 2) finding the average c.v. for the four identical spots in the four identical blocks. When comparing the c.v. of the four identical spots, intensity data with a mean value less than 500 units was ignored (for the purpose decreasing the effects of negative c.v. values and small numbers on the final average c.v.). Slide 3 performed too poorly to be analyzed. The signals measured for slide 5 were much lower than for the other slides. Only the strongest signals were above the cutoff of 500 and were included. Graphs illustrating plots of the signal intensity from Cy3 versus Cy5 from each spot for slides 1, 2 and 4 are shown in FIGS. 4A, 4B and 4C, respectively.

TABLE 3

| Slide No. | Slide Type | r² (Cy3 vs Cy5) | % c.v. Cy3 | % c.v. Cy5 |
|---|---|---|---|---|
| 1. | Dendrite | 0.985 | 19.6 | 18.3 |
| | (PAMAM) | | | |
| 2. | Corning | 0.972 | 34.1 | 33.6 |
| | CMT-GAPS | | | |
| 3. | APS | N/C | N/C | N/C |
| 4. | PLL | 0.930 | 20.9 | 27.8 |
| | Cel Associates | | | |
| 5. | PLL (Giaman) | 0.921 | 17.6^{c} | 21.5^{c} |

### EXAMPLE 7

### Decoration of Aminated Surfaces with Activatible Groups

Aminated slides prepared according to Example 2 were acylated with the NHS ester of an arylazide. 20 mg of ANB-NOS (N-5-azido-2-nitrobenzoyloxysuccinimide, Pierce Chemicals, Rockford, Ill.) was dissolved in 50 mL DMF and was transferred to a reactor that was sufficient for the containment of 5 slides. Five microscope slides were dropped into the solution and the container was sealed with a tight fitting cap. The solution was agitated by inversion for a few minutes and allowed to incubate for 2 hours at room temperature. The DMF solution was decanted from the slides, replaced with fresh DMF and the solution was agitated for circa 2 minutes. This process was repeated 4 times, after which the slides were transferred to a microscope slide rack and washed twice with water. The slides were dried by centrifugation and stored at −20° C.

### EXAMPLE 8

### Addition of PEG Spacer to the APS Coated Surface

Aminated slides prepared according to Example 2 were acylated by the protocol described in Example 7 with poly(ethylene glycol)-α-N-hydroxysuccinimidylpropionate, β-maleimide (MW=3400, Shearwater Polymers, Huntsville, Ala.) or α-vinyl sulfone, ω-N-hydroxysuccinimidyl ester of poly(ethylene glycol)-propionic acid (MW=3400, Shearwater Polymers, Huntsville, Ala.). In this case, the slides were incubated with the activated ester for 24 hours.

### EXAMPLE 9

### Measurement of the Density of Functionalization

The density of functionalization was measured by the method of Guar et al (Guar, R. K.; Gupta, K. C. Analytical Biochemistry 1989 180, 253-258) that relies on the high molar extinction coefficient of the dimethoxytrityl (DMT) cation. Sulfo SDTB (Sulfosuccinimidyl-4-o-(4,4′-dimethoxytrityl) butyrate, Pierce Chemicals, Rockford, Ill., 50 mg) was dissolved in 50 mL DMF. 25 mL was transferred to a plastic container and the amine modified microscope slides were added. The containers were sealed and agitated briefly. After 2 hours at room temperature, the solution was discarded and the slides were washed four times with DMF and the slides were transferred to a microscope slide rack. The slides were washed twice in water and dried in the centrifuge. The number of dimethoxytrityl groups was then measured by cleavage of the trityl ether with 30% perchloric acid. Each slide to be analyzed was fitted with a “Secure Seals™” chamber (SA500 from Grace Bio-Lab, Bend, Oreg.) and was incubated with 30% perchloric acid for 15 hours. The absorbance of the perchloric acid solution was measured at 498 nm and the concentration of the DMT was calculated from the absorbance.

Absorbance from the DMT cation could not be detected in any case within the error of the experiment (0.01 absorbance units), placing the concentration of trityl on the surface below 15990 molecules per micron². Since all primary amines on the surfaces tested are expected to react with the NHS ester that carries the DMT ether, the concentration of primary amino groups on the surface must be below 15990 per micron².

Since fluorescence could be measured from the amino-modified surfaces after acylation with a Rhodamine NHS-ester, it is possible to measure the concentration of acylated amines by calibrating the fluorescence scanner using Rhodamine standards (TAMRA labeled oligo, Synthetic Genetics, 5′-TAMRA-TCGAATAGTATCCTGGT). A known volume of each standard solution (45 μL) was placed on a sheet of glass and covered with a standard microscope slide (Corning, 25 mm×75 mm) and the glass plate was scanned for fluorescence. The concentrations of the standards were: 800 nM, 160 nM, 32 nM, 6.4 nM, 1.3 nM, 0.256 nM). The buffer used for dilution of the standard oligos was 50 mM Tris (pH=8). The volume of solution was chosen such that the entire surface of the microscope slide would be wet with solution. The slides to be assayed (i.e. those that had been acylated with Rhodamine) were placed face down onto 45 μL of the buffer used to prepare the standards next to the slides that covered the standards. The average fluorescence signal measured from each standard and slide was calculated from the fluorescent images using as is described in Example 3. From the average values of fluorescence from the standards, a calibration plot was constructed and the relationship between the fluorescence intensity and the number of fluorescent molecules per square micron was determined to be: y=0.247 X−184.7, where y=number of fluorescent molecules and x=average fluorescence intensity.

A number of implementations of the invention have been described. Nevertheless, it will be understood that various modifications may be made without departing from the spirit and scope of the invention. Accordingly, other embodiments are within the scope of the following claims.

What is claimed is:

1. A microarray substrate fabrication method, comprising:
providing a substrate having a surface;
exposing the substrate to a concentration of linker molecules in the gas phase under conditions sufficient to couple a plurality of the linker molecules to the substrate surface;
and exposing the substrate to a concentration of one or more spacer molecules, each of the spacer molecules including one or more attachment sites for coupling a biological receptor to the surface, the substrate being exposed to the concentration of spacer molecules under conditions sufficient to couple one or more spacer molecules to each of a plurality of the coupled linker molecules to form a functionalized substrate surface.

2. The method of claim 1, wherein:
the functionalized substrate surface has a uniformity of coverage with attachment sites, the uniformity of coverage having a coefficient of variance of less than about 0.25 when the uniformity of coverage is determined by:
exposing the functionalized substrate surface to a concentration of fluorescent reporter molecules under conditions sufficient to couple a plurality of the fluorescent reporter molecules to a plurality of the attachment sites;
exciting the fluorescent reporter molecules coupled to the attachment sites and obtaining a fluorescent emission image of the excited fluorescent reporter molecules, the fluorescent emission image including a plurality of pixels corresponding to locations on the functionalized substrate surface, each pixel in the fluorescent emission image having a pixel value; and
calculating the uniformity of coverage from the fluorescent emission image by calculating the coefficient of variance of the pixel values in the image.

3. The method of claim 2, wherein:
the uniformity of coverage has a coefficient of variance of less than about 0.20.

4. The method of claim 2, wherein:
the uniformity of coverage has a coefficient of variance of less than about 0.15.

5. The method of claim 1, wherein:
the linker molecules include a functionalized alkyl silane.

6. The method of claim 1, wherein:
the linker molecules include a silane comprising one or more functional groups selected from the group consisting of alkyl halide, amino, thiol, glycidyl, alkene, alkyne, carboxyl, aldehyde, hydrizide, hydroxyl, aryl or heteroaryl.

7. The method of claim 1, wherein:
the linker molecules are coupled to the substrate surface through one or more covalent bonds.

8. The method of claim 1, wherein:
the spacer molecules include a Starburst® dendrimer.

9. The method of claim 6, wherein:
the Starburst® dendrimer is a polyamine.

10. The method of claim 1, wherein:
the spacer molecules include a polyethylene glycol.

11. The method of claim 1, wherein:
the spacer molecules include a spacer molecule selected from the group consisting of dendrimers, polyethylene glycols, polyacrylic acid and other vinyl polymers, deoxyribonucleic acids or ribonucleic acids, and amino acid homopolymers.

12. The method of claim 1, wherein:
one or more of the spacer molecules have a linear structure.

13. The method of claim 1, wherein:
one or more of the spacer molecules have a branched structure.

14. The method of claim 1, wherein:
one or more of the spacer molecules have a dendritic structure.

15. The method of claim 1, wherein:
one or more of the attachment sites are provided by a functional group selected from the group consisting of amines, amides, esters, ethers, thioethers, alkyls, alkenyls, alkynyls, aryls and heteroaryl.

16. The method of claim 1, wherein:
the spacer molecules include a spacer molecule having a plurality of electrostatic sites for attracting a biological receptor to the surface.

17. The method of claim 16, wherein:
the spacer molecules include a histone.

18. The method of claim 16, wherein:
the spacer molecules include a Starburst® polyamidoamine Generation 4 dendrimer.

19. The method of claim 1, wherein:
the spacer molecules are coupled to the linker molecules through one or more covalent bonds.

20. The method of claim 1, further comprising:
covalently coupling an activating group to each of a plurality of attachment sites.

21. The method of claim 20, wherein:
the activating group is a photoactivating group.

22. The method of claim 20, wherein:
the activating group is an azide containing functional group.

23. The method of claim 20, further comprising:
exposing the substrate to a plurality of biological receptors; and
activating the activating group to attach a plurality of the biological receptors to the attachment sites.

24. A functionalized microarray substrate prepared by the method of claim 1.

25. The functionalized microarray substrate of claim 21, wherein:
the functionalized substrate surface has a uniformity of coverage with attachment sites, the uniformity of coverage having a coefficient of variance of less than about 0.25 when the uniformity of coverage is determined by:
exposing the functionalized substrate surface to a concentration of fluorescent reporter molecules under conditions sufficient to couple a plurality of the fluorescent reporter molecules to a plurality of the attachment sites;
exciting the fluorescent reporter molecules coupled to the attachment sites and obtaining a fluorescent emission image of the excited fluorescent reporter molecules, the fluorescent emission image including a plurality of pixels corresponding to locations on the functionalized substrate surface, each pixel in the fluorescent emission image having a pixel value; and
calculating the uniformity of coverage from the fluorescent emission image by calculating the coefficient of variance of the pixel values in the image.

26. The functionalized microarray substrate of claim 25 wherein:
the uniformity of coverage has a coefficient of variance of less than about 0.20.

27. The functionalized microarray substrate of claim 25, wherein:
the uniformity of coverage has a coefficient of variance of less than about 0.15.

28. A microarray prepared by the method of claim 23.

29. A microarray substrate fabrication method, comprising:
providing a substrate having a surface;
exposing the substrate to a concentration of linker molecules under conditions sufficient to couple a plurality of the linker molecules to the substrate surface; and
exposing the substrate to a concentration of one or more Starburst™ Dendrite spacer molecules under conditions sufficient to couple one or more spacer molecules to each of a plurality of the coupled linker molecules to form a functionalized substrate surface.

30. A microarray substrate fabrication method, comprising:
providing a substrate having a surface;
exposing the substrate to a concentration of linker molecules under conditions sufficient to couple a plurality of the linker molecules to the substrate surface; and
exposing the substrate to a concentration of one or more polyethylene glycol spacer molecules under conditions sufficient to couple one or more spacer molecules to each of a plurality of the coupled linker molecules to form a functionalized substrate surface.

31. A microarray substrate comprising:
a substrate surface;
a plurality of linkers coupled to the substrate surface; and
a plurality of spacers, each spacer being coupled to one or more linkers and including one or more attachment sites for coupling a biological receptor to the substrate surface, the microarray substrate having a uniformity of coverage with attachment sites, the uniformity of coverage having a coefficient of variance of less than about 0.25 when the uniformity of coverage is determined by:
exposing the functionalized substrate surface to a concentration of fluorescent reporter molecules under conditions sufficient to couple a plurality of the fluorescent reporter molecules to a plurality of the attachment sites;
exciting the fluorescent reporter molecules coupled to the attachment sites and obtaining a fluorescent emission image of the excited fluorescent reporter molecules, the fluorescent emission image including a plurality of pixels corresponding to locations on the functionalized substrate surface, each pixel in the fluorescent emission image having a pixel value; and
calculating the uniformity of coverage from the fluorescent emission image by calculating the coefficient of variance of the pixel values in the image.

32. The microarray substrate of claim 31, wherein:
the uniformity of coverage has a coefficient of variance of less than about 0.20.

33. The microarray substrate of claim 31, wherein:
the uniformity of coverage has a coefficient of variance of less than about 0.15.

34. The microarray substrate of claim 31, wherein:
the linkers are coupled to the substrate surface through one or more covalent bonds.

35. The microarray substrate of claim 34, wherein:
the spacers are coupled to the linkers through one or more covalent bonds.

36. The microarray substrate of claim 31, further comprising:
a plurality of activating groups, each activating group being coupled to one of the attachment sites.

37. The microarray substrate of claim 31, wherein:
the linkers are derived from one or more alkyl silanes.

38. The microarray substrate of claim 37, wherein:
the linkers include a silane comprising one or more functional groups selected from the group consisting of alkyl halide, amino, thiol, glycidyl, alkene, alkyne, carboxyl, aldehyde, oxime, hydrizide, and hydroxyl.

39. The microarray substrate of claim 31, wherein:
the spacers are derived from one or more Starburst® dendrimers.

40. The microarray substrate of claim 39, wherein:
the Starburst® dendrimers are polyamines.

41. The microarray substrate of claim 31, wherein:
the spacers are derived from polyethylene glycol.

42. The microarray substrate of claim 31, wherein:
the spacers include a spacer molecule selected from the group consisting of dendrimers, polyethylene glycols, deoxyribonucleic acids or ribonucleic acids, and amino acid homopolymers.

43. The microarray substrate of claim 31, wherein:
the spacers include a plurality of electrostatic sites for attracting a biological receptor to the surface.

44. A microarray substrate, comprising:
a substrate surface;
a plurality of alkylsilane linkers coupled to the substrate surface; and
a plurality of Starburst™ Dendrite spacers coupled to the alkylsilane linkers.

45. A microarray, comprising:
a microarray substrate according to claim 44; and
a plurality of biological receptors coupled to a plurality of the Starburst™ Dendrite spacers.

46. A microarray substrate, comprising:
a substrate surface;
a plurality of alkylsilane linkers coupled to the substrate surface; and
a plurality of polyethylene glycol spacers coupled to the alkylsilane linkers.

47. A micro array, comprising:
a microarray substrate according to claim 46; and
a plurality of biological receptors coupled to a plurality of the polyethylene glycol spacers.
